(19) United States
(12) Patent Application Publication
(10) Pub. No.: US 2026/0066137 A1
(43) Pub. Date: Mar. 5, 2026

(54) SLOWING THE PROGRESSION OF AN ADVERSE HEALTH CONDITION USING REFLECTION THROUGH A DIGITAL COMMUNICATION NETWORK

(71) Applicant: Sequelae, Inc., Minneapolis, MN (US)

(72) Inventors: Sara Taylor, Edina, MN (US); Scott Archibald, Wayzata, MN (US); Benjamin C. Wiegand, Yardley, PA (US); Shane Hoversten, Brooklyn Park, MN (US); Steven J. Catani, Athens, GA (US)

(73) Assignee: Sequelae, Inc., Minneapolis, MN (US)

(21) Appl. No.: 19/313,469

(22) Filed: Aug. 28, 2025

Related U.S. Application Data
(60) Provisional application No. 63/688,118, filed on Aug. 28, 2024.

(51) Int. Cl. G16H 80/00; G16H 15/00

(52) U.S. Cl. CPC G16H 80/00; G16H 15/00

(57) ABSTRACT

Methods and systems for flowing a progression of an adverse health condition using reflection through a digital communication network (DCN) are provided. A DCN is provided for a plurality of members of a population to communicate with each other and communications from an individual member are analyzed. A time to transmit an automated communication to the individual member is determined and the automated communication is transmitted to the individual member at the determined time. The automated communication requests that the individual member respond indicating a current level of at least one of a psychological or physiological measurement. A response from the individual member is received through the DCN.

### CROSS-REFERENCE TO RELATED APPLICATIONS

This application claims the benefit of U.S. Application No. 63/688,118, filed on Aug. 28, 2024, which is incorporated herein by reference in its entirety.

### BACKGROUND

Various embodiments relate generally to healthcare systems, methods, devices, and computer programs and, more specifically, relate to slowing a progression of an adverse health condition using reflection through a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

People take actions—whether simple, hard, good, or bad, etc.—that impact their health all the time. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences, thus it is desirable for people to reflect their actions and potential consequences.

Computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities. Reflecting takes conscience effort and is often emotionally draining and cognitively draining. Thus, a person may find it easier to not reflect on such actions.

Thus, what is needed is a way to incentivize user reflection of their own psychological or physiological measurement through a digital communication network.

### SUMMARY

Example aspects of the present disclosure include:

A method to slow progression of an adverse health condition of an individual member of a population according to at least one embodiment of the present disclosure comprises providing a digital communication network (DCN) for a plurality of members of a population to communicate with each other; analyzing communications from an individual member in the DCN; determining a determined time to transmit an automated communication to the individual member; transmitting the automated communication to the individual member at the determined time, the automated communication requesting that the individual member respond indicating a current level of at least one of a psychological or physiological measurement; and receiving a response from the individual member through the DCN.

Any of the aspects herein, wherein the automated communication is transmitted to the individual member two or more times each day and one or more additional responses are received.

Any of the aspects herein, wherein the psychological measurement is stress.

Any of the aspects herein, further comprising presenting, to the member, a clickable choice of level of the psychological or physiological measurement in the automated communication.

Any of the aspects herein, wherein the determined time is based on at least one of when the individual member is active on the DCN, a sleep-wake cycle of the individual member, an eating cycle of the individual member, a work cycle of the individual member, or an activity cycle of the individual member.

A method to slow progression of an adverse health condition of an individual member of a population according to at least one embodiment of the present disclosure comprises providing a digital communication network (DCN) for a plurality of members of a population to communicate with each other; receiving at least one of a psychological measurement, a physiological measurement, or one or more levels of a physiological measurement of an individual member; transmitting a communication to the individual member to request the individual member to reflect on a cause of at least one of the one psychological measurement or physiological measurement, or a change in the levels of the physiological measurement; and receiving the reflection from the individual member.

Any of the aspects herein, wherein the communication requests that the individual member reflects over time on the DCN.

Any of the aspects herein, wherein the communication includes one or more questions to illicit the reflection.

Any of the aspects herein, further comprising: receiving one or more communications from the individual member in the DCN; analyzing the one more communications; and generating the one or more questions based on the analysis of the one or more communications.

Any of the aspects herein, further comprising: repeatedly sending additional questions to illicit reflection on factors correlating to the at least one psychological measurement, physiological measurement, or the change in the levels of the physiological measurement; and receiving additional response to the additional questions on the DCN.

Any of the aspects herein, wherein the reflection comprises at least one of a measurement derived from a wearable device, a measurement derived from lab analysis of a sample provided by the individual member.

Any of the aspects herein, wherein the at least one psychological measurement, physiological measurement, or levels of the physiological measurement are received from observing an occurrence of the at least one psychological measurement and/or physiological measurement.

Any of the aspects herein, further comprising observing at least one additional occurrence of the physiological measurement.

Any of the aspects herein, wherein observing the physiological measurement comprises observing a level of a physiological measurement.

Any of the aspects herein, wherein the at least one psychological measurement, physiological measurement, or levels of the physiological measurement are received as a report from the individual member.

A method to slow progression of an adverse health condition of an individual member of a population according to at least one embodiment of the present disclosure comprises providing a digital communication network (DCN) for a plurality of members of a population to communicate with each other; generating a report of a level of a psychological measurement from an individual member over time in a graphical format; displaying the report to the individual member; requesting the individual member to reflect on causes of the level of the psychological measurement over time on the DCN; and receiving a reflection from the individual member on the DCN.

Any of the aspects herein, further comprising: receiving an updated level of the psychological measurement from the individual member over an updated time; updating the report of the level of the psychological measurement in the graphical formal based on the updated level of the psychological measurement and the reflection; and displaying the updated report to the individual member.

Any of the aspects herein, wherein the reflection is at least one of textual, verbal, a verbal communication converted to a textual communication, a visual communication, or a graphical communication.

Any of the aspects herein,, wherein the report is generated by the individual member.

Any of the aspects herein, further comprising receiving an annotation on the graphical format of the level of the psychological measurement over time.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4 is a logic flow diagram that illustrates the operation of a method, in accordance with one or more embodiments.

FIG. 5A shows an example of a computing system, in accordance with one or more embodiments.

FIG. 5B shows an example of a network, in accordance with one or more embodiments.

### DETAILED DESCRIPTION

Medical interventions, such as like pharmaceuticals, can be used over the long term to mitigate the consequence, severity, or discomfort associated with a disease. In most cases this is the intended use, at least until the underlying disease progresses to the state where an even more powerful intervention may be recommended to a person. This can often be the case even when the disease has a basis in the person's lifestyle as patients and manufacturers may view the pharmaceuticals as a replacement to making lifestyle changes which can be harder to adopt. Finding and adopting lifestyle changes that work is a cognitively and emotionally challenging process that is incompatible for most people while dealing with the underlying disease and its symptom at the same time.

Interventions can be used in ways to allow short-term use of the intervention to give the patient cognitive and emotional room to better appreciate that lifestyle change is possible (and preferred over a lifetime of pharmaceutical use). However, people can seize upon the benefits of the intervention if they take the time to consider their conditions more fully. Various embodiments are directed to incentivize user reflection using a digital communication network (DCN) as reflecting on “what” the use of the intervention helped overcome in a person, lifestyle, and environment can allow the intervention to be used as a tool rather than a crutch. Enabling a user to earn incentives for lifestyle promoting activities, and then use those incentives to obtain other therapies can help incentivize the user to engage in those activities. Additionally, embodiments may be used to slowing progression of an adverse health condition or to manage a disease.

Various embodiments are directed to inducing reflection in an individual member of a population within a digital communication network (DCN). Reflections are an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

Reflection may be considered as part of the change process—a person does or observes something, and then reflects on the action, or observation, and the consequences. This process implies some degree of planning, either in adding reflection to a planned action, or scheduling periods to reflect (these can be formal as in professional counseling). Unfortunately, much of life is unplanned and unobserved.

Various embodiments overcome those problems, for example, by sending an automated communication to an individual member requesting that they indicate their current level of a psychological measurement such as, for example, their level of stress. The act of indicating their level of a psychological measurement helps build more awareness of the member's psychological state and its connection to their lifestyle.

The DCN may also follow-up on the communications by requesting additional reflection from the member. For example, the DCN may ask the member to reflect on the factors that caused their response to the automated communication. The member may respond to the follow-up request which may lead to further communications.

In some embodiments, the DCN, using a conversation bot, is able to help patients with seeking help for their disease or other health related issue. The conversation bot, using artificial intelligence (AI), can review community discussions on the DCN to identify key issues that may be a concern for an individual member. The bot can ask questions of the individual member so that they can reflect on their situation and/or better appreciate the consequences of any actions. This can help identify conditions, reactions, etc. The conversation bot can also review community discussions to identify possible questions that can, for example, be asked of a doctor to help a patient (e.g., the individual member) with the preliminary work and setting them up to get the key information for proper medical intervention and treatment.

Various embodiments also use AI-driven bots to induce more user reflections. In traditional systems, conversation bots respond to questions with community or expert-sourced answers whereas in embodiments of the present disclosure the conversation bots respond to observations (which may be provided directly or indirectly by the user) with questions designed to cause user reflection on those observations. User reflections may be used by the conversation bot to pinpoint better information for the user, for example, by providing more applicable community or expert-sourced answers. In other embodiments, the individual member's reflections may be provided to care givers and/or physicians in order to better evaluate the individual member.

This may be based in part on creating greater awareness of lifestyle changes and medical options. A lifestyle (or behavior) change can be evaluated based on an individual member's reflections. Such induced reflections may be used to help identify the user's condition, for example, to evaluate whether a lifestyle change is working.

Additionally, the individual member's reflections can be used to provide the individual member's experience as a social aspect. As a social process, value can be placed on those members of a community that have relevant experience. The reflections from the individual members can be induced in order to help elevate the aspects of the experience that may be more instructional to the community as a whole. This can help increase the number of members of the community thinking about an action, for example, creating an atmosphere where people can learn about others who have undergone a change they are considering.

Additionally, the community can be used to help support the individual member in other ways. Lifestyle interventions are inherently safe. They use the philosophy that any action now is preferred to a “better” action later. They also support the concept that ideas and communication are health care.

The community can also be used to encourage lifestyle interventions, which are inherently safe. Such a community may use the philosophy that any action now is preferred to a “better” action later and also support the concept that ideas and communication are health care. Conventionally, lifestyle change has been looked at as an individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do,” which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual member or members of a population. For example, in a community where a person is a peer, the actions they take and learn from are that their volition and may result in increased agency (or autonomy) or self-efficacy. This not only increases the chances of continuous lifestyle improvement, but improved outcomes throughout the health system.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Online communities also give access to information and guidance to help patients discover the healthy actions that may work best for them. Members can also find a community of patients and others who can support them and who they can support through online or arranged in-person conversations. Using their personal computers or devices (such as a cell phone), patients can have access to activity trackers, health testing, etc., as well as other tools aimed at improving their health. Such information may be gathered from connected devices, such as, glucose measurements from a continuous glucose monitor, and wearables like FITBIT®. Patients and doctors can also communicate to address issues like food, transportation, and other factors that impact their health.

In many situations, patients can use their personal devices, such as a computer, tablet, cell phone, etc., to interact with a host system on a server. The host system can provide a platform for relaying communications, such as public posts, etc., and/or direct communications. Additionally, the host system can store patient/user information, for example, biometric information, test results, personal data, etc. In some embodiments, the host system may provide services/apps, e.g., monitoring or even games.

Thus, it is desirable to provide systems and methods that induce reflection in an individual member regarding their level of a psychological or physiological measurement whether through the DCN directly or via one or more conversation bots. Such reflections can beneficially provide insight into the individual member's life and lifestyle and whether certain actions or interventions are beneficial to the individual member.

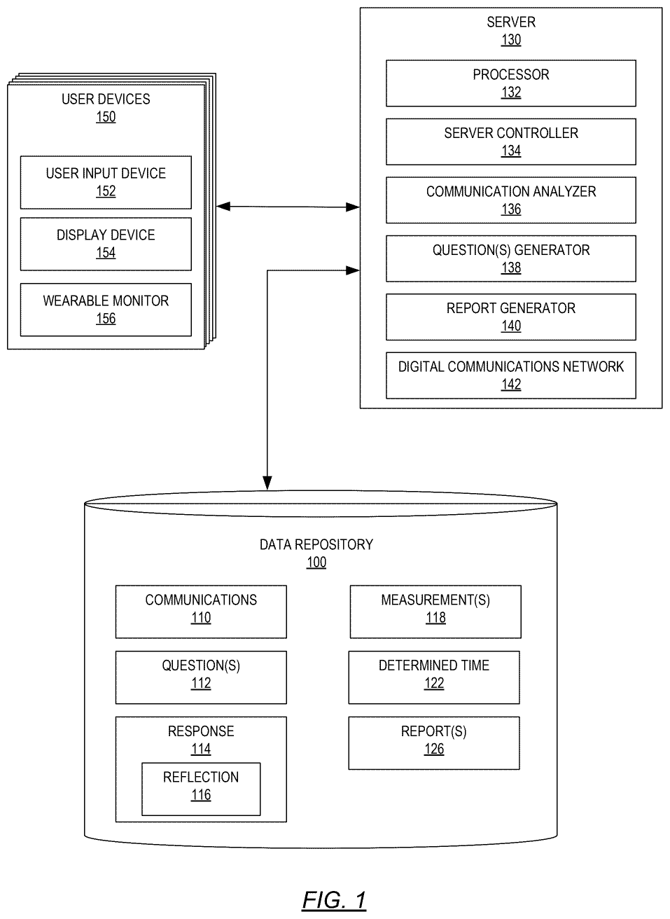

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communication(s) (110). The communications (110) are communications from members in a digital communications network (DCN) (142) or from the DCN (142) (e.g., the DCN as a chatbot). The communications (110) may be, for example, text-based, image-based, multimedia communications, audio communications, or any combinations thereof. The communications (110) may be between members, communications in a forum, communications with the DCN (142), or communications generated by the DCN (142) (e.g., communications from a chatbot). The communications (110) can be one-to-one communications, one-to-many communications, one-to-system communications, system-to-one communications, and system-to-many communications. The communications (110) can also include one or more reflections (116) from a member such as an individual member.

The communication (110) can be generated by the DCN (142) and sent to a member. The communication (110) can include, for example, a request for an individual member to respond with a current level of a psychological or physiological measurement from the individual member. In other instances, the communication (110) can include a request for the individual member to reflect on a cause of their psychological or physiological measurement, a level of their psychological or physiological measurement, and/or a change in the level of their psychological or physiological measurement. In some embodiments, the communications (110) can also include one or more questions to illicit reflection.

The data repository (100) also stores question(s) (112). The questions (112) are questions directed to the individual member and may include yes/no questions or open-ended questions. The questions (112) may be designed so as to illicit reflection from the individual member on their psychological or physiological measurement, levels of their psychological or physiological measurement, and/or changes in their levels of psychological or physiological measurement. The questions (112) can include one question, two questions, or more than two questions.

The data repository (100) also stores response(s) (114). The responses (114) are responses (114) to the questions (112) and the request in the communication (110) received from the individual member. The response (114) may include, for example, a yes/no answer, text, and/or media (e.g., audio, visual, images, videos, etc.). The response (114) may also be a verbal communication, a verbal communication converted to a textual communication, a visual communication, or a graphical communication. The response (114) may be, for example, the psychological or physiological measurement as perceived by the individual member and/or a level of the psychological or physiological measurement.

The response (114) may also be a reflection (116) from the individual member. The reflection (116) may be a reflection (116) on the psychological or physiological measurement, the level of the psychological or physiological measurement, a change in the level of the psychological or physiological measurement, or a cause of the psychological or physiological measurement, the level of the psychological or physiological measurement, or the change in the level of the psychological or physiological measurement. The reflection (116) may also include a measurement derived from a wearable device and/or a measurement derived from lab analysis of a sample provided by the individual member.

The data repository (100) also stores measurement(s) (118). The measurements (118) may be received at discrete time periods, or may be received over time. The measurements (118) may be, for example, the measurement derived from a wearable device and/or a measurement derived from lab analysis of a sample provided by the individual member. In some embodiments, the measurement (118) may be obtained from receiving a response such as the response (114) from the individual member that includes the measurement (118). In other embodiments, the measurement (118) may be received as input from the individual member to a clickable choice of level of the psychological or physiological measurement. The measurement (118) may also be received in a report (126) provided by the individual member.

The measurement (118) may also be a measurement of the psychological or physiological measurement. The psychological measurement may be, for example, stress. Such measurement (118) may be obtained, for example, from observing an occurrence of the psychological or physiological measurement. For example, the physiological measurement may be a physiological measurement as a biomarker derived from one of: saliva, blood, breath, urine, stool, and/or a wearable device.

The data repository (100) also stores a determined time (122). The determined time (122) is a time or time period where a communication (110) to the individual member is to be sent to the individual member. The time period is based on, for example, when an individual member is active on the DCN, a sleep cycle of the individual member, an eating cycle of the individual member, a work cycle of the individual member, or an activity cycle of the individual member. Such determined time (122) helps ensure that the individual member would be more willing or motivated to respond to the communication (110). For example, the determined time (122) may be immediately after the individual member has finished an activity. Such timing may incentivize the individual member to respond to the communication (110) to, for example, provide their psychological or physiological measurement.

The data repository (100) also stores report(s) (126). The report (126) may be received from the individual member. The report (126) may include, for example, a report on a level of a psychological or physiological measurement.

In other instances, the report (126) may be generated by the DCN (142) and transmitted to the individual member. In such instances, the report (126) may include the level of the psychological or physiological measurement over time and shown in a graphical format. It will be appreciated that in other embodiments, the report (126) may be shown in any other format.

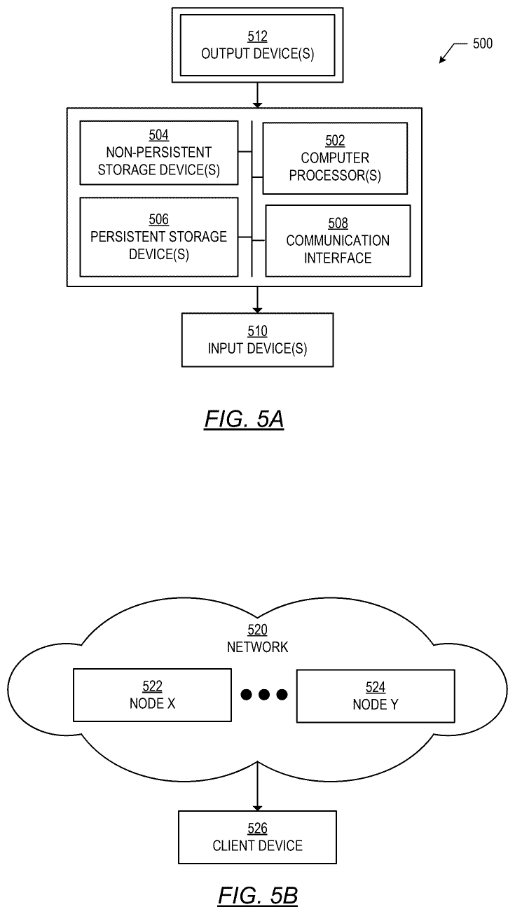

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136), a question(s) generator (138), and/or a report generator (140). An example of a computer system and network that may form the server (130) is described with respect to FIG. 5A and FIG. 5B.

The server (130) also includes a computer processor (132). The computer processor (132) is one or more hardware or virtual processors which may execute computer readable program code that defines one or more applications, such as the communication analyzer (136), the question(s) generator (138), and/or the report generator (140). An example of the computer processor (132) is described with respect to the computer processor(s) (502) of FIG. 5A.

The server (130) also may include a server controller (134). The server controller (134) is software or application specific hardware which, when executed by the computer processor (132), (502), controls and coordinates operation of the software or application specific hardware described herein. Thus, the server controller (134) may control and coordinate execution of the communication analyzer (136), the question(s) generator (138), and/or the report generator (140).

The server (130) also includes a communication analyzer (136). The communication analyzer (136) is software or application specific hardware which, when executed by the computer processor (132), receives the communications (110) from an individual member as input and can determine the determined time (122) based on at least the communications (110). The communication analyzer (136) can also generate communications (110) for the DCN (142) to send to members of the population requesting, for example, the measurements (118), and/or the response (114), and/or asking the questions (112).

The server (130) may also include the question(s) generator (138). The question(s) generator (138) is software or application specific hardware which, when executed by the computer processor (132), receives the communications (110) from the individual member and generates the one or more questions (112). The question(s) generator (138) may determine one or more factors about the individual member from the communication (110) that the individual member may more favorably respond to in order to illicit a response (114) from the individual member.

The server (130) may also include the report generator (140). The report generator (140) can receive, for example, the measurements (118), communications (110), and/or response(s) (114) from the individual member to generate the report (126).

The server (130) also includes a digital communications network (DCN) (142). The DCN is a network through which members of a population can interact or communicate with each other, or with a system supported by the DCN.

The DCN (142) can analyze communications that are received as text (or audio) which is received via streaming or in batches. The DCN may analyze the communication individually or as a part of a larger conversation. Machine learning (ML) models, or natural language processing (NLP) algorithms may be used to examine the communication (for example, counting frequency of certain words). The DCN (142) produces an output which is used to update the system's understanding of how well someone is doing at reflection.

The DCNs (142) can also manage a member's communication feed to prioritize communications which contain ideas that might be applicable to them. This can be based on various factors, such as the patient's own biological information, the individuals they follow in the network, etc.

The DCN (142) may be, for example, an artificial intelligence bot configured to derive generated communications from analysis of existing communications (110) in the DCN (142) or biometrics provided to the DCN (142). Biometric data can be used in many instances such as the provisioning and commissioning of health care. For example, biometric data can be used to diagnose diabetes, prescribe hypertension medication when a patient's blood pressure exceeds a certain value, prescribe cancer drugs, etc. In another example, cancer drugs can be available for reimbursement if a certain genetic signature is present in the tumor. The decision tree and other logic behind these relationships considers many things, including what is known about the variance in the intervention's efficacy, therapeutic index, the accuracy and precision in the biometric data's measurement, as well as the cost-benefit ratio of the intervention compared with other interventional options.

The biometric data, as well as reflections from the individual member, can be used in a “feedback” decision tree where, for example, an intervention can be prescribed or recommended to an individual member based on the biometric data and/or the reflections. Then the biometric data or additional reflections can be obtained to study the effects or results of the intervention.

In some embodiments, the DCN (142) uses a conversation bot to help patients with seeking help for medical conditions. The conversation bot is an artificial intelligence (AI) tool that can review community discussions on the DCN (142) to, for example, identify key issues that may be a concern for a patient. The bot can ask questions of the individual member so that they can reflect on their situation and/or better appreciate the consequences of any actions. This can help identify conditions, reactions, etc. The bot can also review community discussions to identify possible questions that should be asked of a doctor to help a patient with the preliminary work and setting them up to get the key information for proper medical intervention and treatment.

In some embodiments, the AI bot may use a visual or graphical interface, for example, a rendered avatar, or a plot or image from the bot or sharing one with the bot. The DCN (142) can send a visual representation showing that person's data from a wearable device, their interaction with the community, their consistency in talking with the bot, etc. The AI bot may then ask questions like “What do you notice?” or “Is anything here surprising?” in order to induce a reflection from the person.

The conversation bot can analyze data from various sources, in part, to formulate its communications with the user. These sources include data from i) the DCN (142), ii) the ongoing conversation with the user, and iii) previous conversations with the user. The conversation bot can also use temporal and situational cues to formulate its communication when next with the member. When analyzing the data, the conversation bot may quote sections of the conversations for use with communications between the user and/or the conversation bot may identify patterns in the discussions. When formulating the communications, the conversation bot can analyze exchanges in the DCN (142) from other members. These communications can be limited to specifically target individuals, such as those deemed to be close contacts to the user or members that are considered influencers.

One-to-one communications with the conversation bot can be triggered and informed by one-to-many communications in the DCN (142), and conversations with the conversation bot can (a) refer individuals to the relevant communications in the DCN (142), (b) suggest posts to the DCN (142), and (c) make reference to ideas contained in the posts. Having two ways to interact can behave in a synergistic way and allow the conversation bot to include a “human in the loop”in a surprisingly scalable way.

During the communication between the user and the conversation bot, the DCN (142) can analyze the user's responses. Based on the input from the user, the DCN (142) may identify an adverse health condition for the user. The conversation bot may then gather more information regarding the adverse health condition, for example, to develop more details to confirm the adverse health condition. The communication may be based on related experiences from another member of the population. The conversation bot may quote content from a post by that other member.

The synergy between the conversation bot and the DCN (142) can facilitate reflection on a topic. Alternatively, this can be seen to be reversed where the act of facilitating reflection synergizes with usage of the bot in the DCN (142).

Using analysis of the user's communications in the DCN (142), the conversation bot can suggest a reflection on a topic in a one-to-one communication. The suggestion may be made as part of a question to the user. The conversation bot may then focus its conversations with that user on that topic for a period of time (e.g., a number of exchanges, a few minutes, a few days, etc.). The conversation bot can incorporate excerpts of communication from the DCN (142) in its responses. The excerpts of communications may include post titles, post topics, quotes from the body of the communication, and/or post contributors. Also, the excerpts could be summaries of communications on the DCN (142). These summaries may be created automatically by the DCN (142) and/or are based on a topic, a member's communications, or a time period.

The bot may prompt the user to reflect on various classes of action. The action may be active or passive. The action may also be one that was contemplated even if not undertaken.

Two or more conversation bots, each with a different personality or a different role (e.g. facilitate reflections, find useful communications, say good morning and inspire a positive start to a day, handle routine network tasks, provide custom communication summaries), may be provided by the DCN (142). The family of bots could each use conversations between the other bots and the user on the DCN (142) to formulate their responses. When working with a user, the bots may be selected based on the personality of the bot that is expected to work best with that user. The decision may be based on past dealings with the user and/or through analysis of that user's communication on the DCN (142). For example, when attempting to induce a reflection, the bot may use a first personality, then switch to a second personality when helping draft a post regarding that reflection. Accordingly, various embodiments can be used to provide a method to improve the communications of the conversation bot through using authentic experiences of the user in the communication.

In some, non-limiting embodiments, the DCN (142) can include use of a VR environment as an intervention, as an alternative channel for conversation bot conversations, and likewise for digital communication posts and conversations. Thus, the use of VR can facilitate reflection. A conversation bot may be used in VR to facilitate reflection on the interventions in the VR environment. As discussed above, the conversation bot may be given a specific personality or role. In VR, the bot may also be provided with an appearance based on a personality or role. Additionally, data from the VR intervention can be used to inform the reflection dialog.

The DCN (142) can analyze communications that are received as text (or audio) which is received via streaming or in batches. The DCN (142) may analyze the communication individually or as a part of a larger conversation. Machine learning (ML) models, or natural language processing (NLP) algorithms may be used to examine the communication (for example, counting frequency of certain words). The DCN (142) produces an output which is used to update the system's understanding of how well someone is doing at reflection.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (500) shown in FIG. 5A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

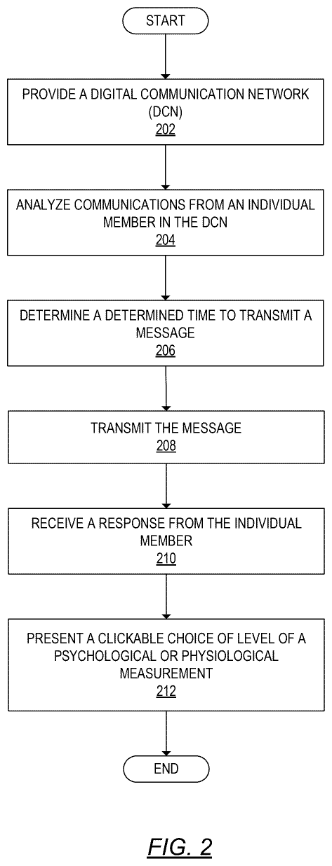

FIG. 2 is a logic flow diagram that illustrates a method, and a result of execution of computer program instructions, in accordance with various embodiments. The method can be used to induce or encourage reflection in an individual member regarding one or more measurements (e.g., psychological measurements, physiological measurements, etc.). The method can also, in other instances, be used in part to facilitate or accomplish at least one of: manage the healthcare cost of the population; reduce the healthcare risk in the population; and/or slow the progression of an adverse health condition or a lifestyle driven disease. The adverse health condition may be, for example, a metabolic disease, an auto-immune disease, or a cardiovascular disease. The disease may be, for example, obesity, diabetes, rheumatoid arthritis, Crohn's disease, psoriasis, eczema, a cardiovascular disease, congestive heart failure, chronic obstructive pulmonary disease, asthma, depression, and anxiety. The adverse health condition may also be a health risk such as, for example, falling or becoming infected with a disease or illness.

At Block 202, a digital communication network (DCN) is provided. The DCN may be the same as or similar to the DCN (142) and provides a network for members of a population to interact with each other or with the DCN.

At Block 204, a step of analyzing communication(s) from an individual member in the DCN is provided. The communications may be the same as or similar to the communications (110) and may be received from an individual member of the members of the DCN. The communications may be analyzed by a communications analyzer such as the communications analyzer (136). The communications may include, for example, key words or phrases indicating various activities or time periods of the individual member. For example, key words or phrases may indicate when the individual member is about to go to sleep, begin an activity, or about to eat. Such analysis can be used to determine a determined time such as the determined time (122), discussed below.

At Block 206, a step of determining a determined time to transmit a communication is provided. The determined time may be used by the DCN to time when a communication to the individual member is automatically sent in order to coincide with the individual member's day. For example, the DCN may analyze the member's communication to determine their sleep cycle, work cycle, eating cycle, etc. The DCN may also evaluate either the content of the communication and/or the timing of the communication.

At Block 208, a step of transmitting the communication is provided. The communication is a communication generated by the DCN to request the individual member to provide their measurement—which may be the same as or similar to the measurement (118). The communication may be transmitted to the individual member through, for example the DCN and may be sent to a user device such as the user device (150).

At Block 210, a step of receiving a response from the individual member is provided. The response from the individual member may be received through the DCN and from, for example, the user device. As previously described, the response may include the measurement.

At Block 212, a step of presenting a clickable choice of level of a psychological or physiological measurement is provided. The clickable choice of level of the psychological or physiological measurement may be presented to the individual member on the user device. It will be appreciated that the Block 212 may occur simultaneously with the Block 208. In other words, the clickable choice may be provided in the communication. In some embodiments, the method may not include the Block 212.

The method described above in FIG. 2 can include more or less steps. The method may also repeat any step or combination of steps.

In at least one example of the above method, the DCN may analyze communications from an individual member to time when a communication to the individual member is automatically sent in order to coincide with the individual member's day. For example, the DCN may analyze the member's communication to determine their sleep cycle, work cycle, eating cycle, etc. The DCN may also evaluate either the content of the communication and/or the timing of the communications.

In such example, the DCN may analyze posts about what the member is eating to determine their eating cycle and a corresponding determined time. The DCN may then time sending a request for the individual member to indicate their level of a psychological measurement at the determined time, which would be a point shortly after the member is expected to have completed a meal. Such determined time may help encourage or incentivize the individual member to provide a response to the DCN.

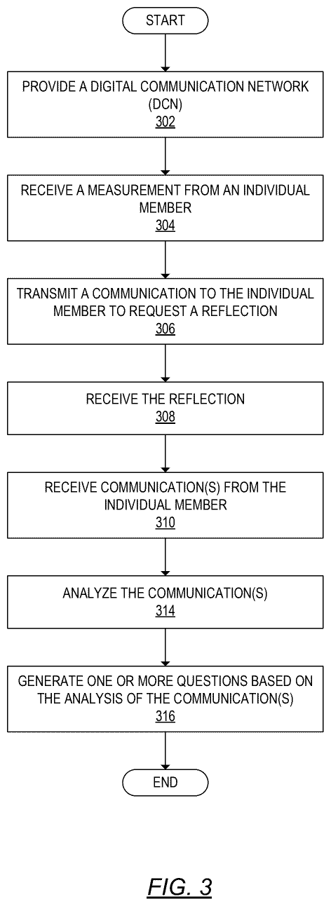

FIG. 3 is a logic flow diagram that illustrates a method, and a result of execution of computer program instructions, in accordance with various embodiments. The method can be used to induce or encourage reflection in an individual member regarding one or more measurements (e.g., psychological measurements, physiological measurements, etc.). The method can also, in other instances, be used in part to facilitate or accomplish at least one of: manage the healthcare cost of the population; reduce the healthcare risk in the population; and/or slow the progression of an adverse health condition or a lifestyle driven disease. The adverse health condition may be, for example, a metabolic disease, an auto-immune disease, or a cardiovascular disease. The disease may be, for example, obesity, diabetes, rheumatoid arthritis, Crohn's disease, psoriasis, eczema, a cardiovascular disease, congestive heart failure, chronic obstructive pulmonary disease, asthma, depression, and anxiety. The adverse health condition may also be a health risk such as, for example, falling or becoming infected with a disease or illness.

At Block 302, a digital communication network (DCN) is provided. The DCN may be the same as or similar to the DCN (142) and provides a network for members of a population to interact with each other or with the DCN.

At Block 304, a step of receiving a measurement from an individual member is provided. The measurement may be the same as or similar to the measurement (118) and may be received at the DCN. The measurement may be, for example, a physiological measurement or a psychological measurement. The measurement may be obtained, for example, by a home test, as a response (114) from the individual member, in a report such as the report (126), or as an observance of an occurrence of the measurement.

At Block 306, a step of transmitting a communication to the individual member to request a reflection is provided. The communication may be the same as or similar to the communication (110) and the reflection may be the same as or similar to the reflection (116). The communication may include, for example, one or more questions such as the one or more questions (112) designed to induce reflection in the individual member.

In a further embodiment, the method also includes repeatedly sending additional questions to illicit reflection on factors and receiving additional responses to the additional questions on the DCN. For example, the questions may be sent to the individual member two or more times in a time period.

At Block 308, a step of receiving the reflection is provided. The reflection from the individual member may be received through the DCN and from, for example, the user device. As previously described, the reflection may include the measurement.

The method may also include the following steps to generate one or more questions.

At Block 310, a step of receiving communication(s) from the individual member is provided. The communications may be received from the individual member via, for example, a user device such as the user device (150). The communication may include the measurements, text, posts, media, etc., from the individual member.

At Block 314, a step of analyzing the communication(s) is provided. The communications may be analyzed by, for example, the communication analyzer (136) to determine one or more factors that may encourage the individual member to provide a reflection to the DCN.

At Block 316, a step of generating one or more questions based on the analysis of the communications is provided. The one or more questions may be generated by, for example, a questions generator such as the questions generator (138). The one or more questions may be designed based on the determined factors to encourage or illicit the reflection from the individual member.

The method described above in FIG. 3 can include more or less steps. The method may also repeat any step or combination of steps.

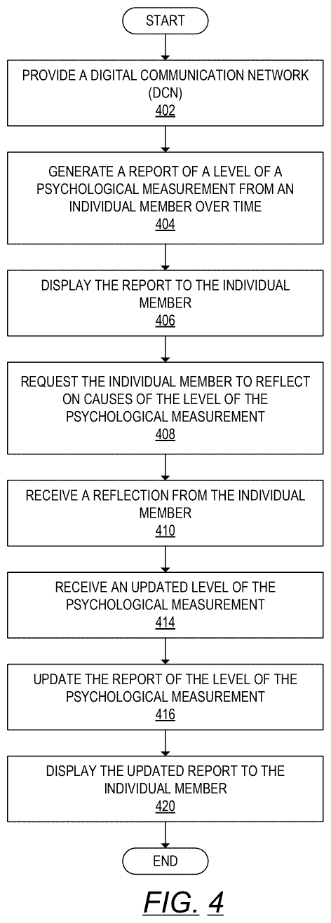

FIG. 4 is a logic flow diagram that illustrates a method, and a result of execution of computer program instructions, in accordance with various embodiments. The method can be used to induce or encourage reflection in an individual member regarding one or more measurements (e.g., psychological measurements, physiological measurements, etc.). The method can also, in other instances, be used in part to facilitate or accomplish at least one of: manage the healthcare cost of the population; reduce the healthcare risk in the population; and/or slow the progression of an adverse health condition or a lifestyle driven disease. The adverse health condition may be, for example, a metabolic disease, an auto-immune disease, or a cardiovascular disease. The disease may be, for example, obesity, diabetes, rheumatoid arthritis, Crohn's disease, psoriasis, eczema, a cardiovascular disease, congestive heart failure, chronic obstructive pulmonary disease, asthma, depression, and anxiety. The adverse health condition may also be a health risk such as, for example, falling or becoming infected with a disease or illness.

At Block 402, a digital communication network (DCN) is provided. The DCN may be the same as or similar to the DCN (142) and provides a network for members of a population to interact with each other or with the DCN.

At Block 404, a step of generating a report of a level of a psychological measurement from an individual member over time is provided. The report may be the same as or similar to the report (126). The report may be shown in a graphical format such as, for example, a graph of the measurement over time, a bar graph indicating the number of times the member has responded with each level, etc. The report may be generated by the individual member or the DCN.

At Block 406, a step of displaying the report to the individual member is provided. The report may be displayed on, for example, a user device such as the user device (150).

At Block 408, a step of requesting the individual member to reflect on causes of the level of the psychological measurement is provided. The reflection requested may be the same as or similar to the reflection (116).

In some embodiments, the DCN may take the form of an artificial intelligence (AI) bot. The AI bot may use a visual or graphical interface, for example, a rendered avatar, or a plot, or image of the bot. The DCN can send a visual representation showing that person's data from a wearable device, their interaction with the community, their consistency in talking with the bot, etc. The AI bot may then ask questions like “What do you notice?” or “Is anything here surprising?” in order to induce a reflection from the person.

At Block 410, a step of receiving a reflection from the individual member is provided. The response from the individual member may be received through the DCN and from, for example, the user device. As previously described, the response may include the measurement.

At Block 414, a step of receiving an updated level of the psychological measurement is provided. The updated level may be provided by the individual member to the DCN.

At Block 416, a step of updating the report is provided. The report may be updated to include the updated level of the psychological measurement over time.

At Block 420, a step of displaying the updated report to the individual member is provided. The updated report may be displayed to the individual member via the user device.

The method described above in FIG. 4 can include more or less steps. The method may also repeat any step or combination of steps.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 5A, the computing system (500) may include one or more computer processor(s) (502), non-persistent storage device(s) (504), persistent storage device(s) (506), a communication interface (508) (e.g., Bluetooth interface, infrared interface, network interface, optical interface, etc.), and numerous other elements and functionalities that implement the features and elements of the disclosure. The computer processor(s) (502) may be an integrated circuit for processing instructions. The computer processor(s) (502) may be one or more cores, or micro-cores, of a processor. The computer processor(s) (502) includes one or more processors. The computer processor(s) (502) may include a central processing unit (CPU), a graphics processing unit (GPU), a tensor processing unit (TPU), combinations thereof, etc.

The input device(s) (510) may include a touchscreen, keyboard, mouse, microphone, touchpad, electronic pen, or any other type of input device. The input device(s) (510) may receive inputs from a user that are responsive to data and communications presented by the output device(s) (512). The inputs may include text input, audio input, video input, etc., which may be processed and transmitted by the computing system (500) in accordance with one or more embodiments. The communication interface (508) may include an integrated circuit for connecting the computing system (500) to a network (not shown) (e.g., a local area network (LAN), a wide area network (WAN) such as the Internet, mobile network, or any other type of network) or to another device, such as another computing device, and combinations thereof.

Further, the output device(s) (512) may include a display device, a printer, external storage, or any other output device. One or more of the output device(s) (512) may be the same or different from the input device(s) (510). The input device(s) (510) and output device(s) (512) may be locally or remotely connected to the computer processor(s) (502). Many different types of computing systems exist, and the aforementioned input device(s) (510) and output device(s) (512) may take other forms. The output device(s) (512) may display data and communications that are transmitted and received by the computing system (500). The data and communications may include text, audio, video, etc., and include the data and communications described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (502), is configured to perform one or more embodiments, which may include transmitting, receiving, presenting, and displaying data and communications described in the other figures of the disclosure.

The computing system (500) in FIG. 5A may be connected to, or be a part of, a network. For example, as shown in FIG. 5B, the network (520) may include multiple nodes (e.g., node X (522) and node Y (524), as well as extant intervening nodes between node X (522) and node Y (524)). Each node may correspond to a computing system, such as the computing system shown in FIG. 5A, or a group of nodes combined may correspond to the computing system shown in FIG. 5A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (500) may be located at a remote location and connected to the other elements over a network.

The nodes (e.g., node X (522) and node Y (524)) in the network (520) may be configured to provide services for a client device (526). The services may include receiving requests and transmitting responses to the client device (526). For example, the nodes may be part of a cloud computing system. The client device (526) may be a computing system, such as the computing system shown in FIG. 5A. Further, the client device (526) may include or perform all or a portion of one or more embodiments.

The computing system of FIG. 5A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium(s), described below, can be used with computer programs written in accordance with the teachings herein, or it may be more convenient to construct a more specialized apparatus to perform the required operations.

The procedures, processes, and/or modules described herein may be implemented in hardware, software, embodied as a computer readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements:

Statement 1. A method to slow progression of adverse health condition of a member of a population, the method comprising: providing a digital communication network (DCN) for individuals of the population to communicate with each other; analyzing communications in the DCN by the member to determine a particular time to send an automated communication; sending the automated communication to the member of the DCN at the particular time, the automated communication requesting that the member respond indicating a current level of a psychological or physiological measurement; and receiving a response from the member through the DCN.

Statement 2. The method of Statement 1 wherein the psychological measurement is stress.

Statement 3. The method of Statement 1, further comprising presenting, to the member, a clickable choice of level of the psychological or physiological measurement in the automated communication.

Statement 4. The method of Statement 1, wherein the automated communication is sent several times per day.

Statement 5. The method of Statement 1, wherein the particular time is based on when the member is active in the DCN.

Statement 6. The method of Statement 1, wherein the particular time is based on a sleep-wake cycle of the member.

Statement 7. The method of Statement 1, wherein the particular time is based on an eating cycle of the member.

Statement 8. The method of Statement 1, wherein the particular time is based on a work cycle of the member.

Statement 9. The method of Statement 1, wherein the particular time is based on an activity cycle of the member.

Statement 10. The method of Statement 1, wherein the adverse health condition is one of: chronic systemic inflammation, malaise, low energy, social dysfunction, and a prodromal disease.

Statement 11. The method of Statement 1, wherein the adverse health condition is a disease.

Statement 12. The method of Statement 11, wherein the disease is one of: obesity, diabetes, rheumatoid arthritis, Crohn's disease, psoriasis, eczema, a cardiovascular disease, congestive heart failure, chronic obstructive pulmonary disease, asthma, depression, and anxiety.

Statement 13. The method of Statement 1, wherein the adverse health condition is a health risk.

Statement 14. The method of Statement 13, wherein the health risk is one of: falling, and becoming infected.

Statement 15. The method of Statement 1, wherein communications in the DCN comprise at least one of: one-to-one communications, one-to-many communications, one-to-system communications, system-to-one communications, and system-to-many communications.

Statement 16. The method of Statement 15, wherein the DCN comprises an AI bot configured to derive communications from analysis of communication in the DCN or from biometrics provided to the DCN.

Statement 17. The method of Statement 1, further comprising sending a request to the member requesting the member reflect on factors causing there response through communication on the DCN.

Statement 18. The method of Statement 17, further comprising receiving a communication on the DCN related to the reflection.

Statement 19. A method to slow progression of adverse health condition of a member of a population, the method comprising: providing a digital communication network (DCN) for individuals of the population to communicate with each other; receiving, from the member, a report on a level of a psychological or physiological measurement in the DCN; sending a request to the member requesting the member reflect on factors causing a response to the level of the psychological or physiological measurement through communications on the DCN; and receiving a communication related to the reflection on the DCN.

Statement 20. The method of Statement 19, wherein the communication related to the reflection is textural.

Statement 21. The method of Statement 19, wherein the communication related to the reflection is a verbal communication.

Statement 22. The method of Statement 21, further comprising translating the verbal communication to a textual communication.

Statement 23. The method of Statement 19, wherein the communication related to the reflection is one of: a visual communication and a graphical communication.

Statement 24. The method of Statement 19, wherein the psychological measurement is stress.

Statement 25. The method of Statement 19, further comprising presenting, to the member, a clickable choice of level of the psychological or physiological measurement.

Statement The method of Statement 19, wherein the adverse health condition is one of: chronic systemic inflammation, malaise, low energy, social dysfunction, and a prodromal disease.

Statement 27. The method of Statement 19, wherein the adverse health condition is a disease.

Statement 28. The method of Statement 27, wherein the disease is one of: obesity, diabetes, rheumatoid arthritis, Crohn's disease, psoriasis, eczema, a cardiovascular disease, congestive heart failure, chronic obstructive pulmonary disease, asthma, depression, and anxiety.

Statement 29. The method of Statement 19, wherein the adverse health condition is a health risk.

Statement 30. The method of Statement 29, wherein the health risk is one of: falling, and becoming infected.

Statement 31. The method of Statement 19, wherein communications in the DCN comprise at least one of: one-to-one communications, one-to-many communications, one-to-system communications, system-to-one communications, and system-to-many communications.

Statement 32. The method of Statement 31, wherein the DCN comprises an AI bot configured to derive communications from analysis of communication in the DCN or from biometrics provided to the DCN.

Statement 33. A method to slow progression of adverse health condition of a member of a population, the method comprising: providing a digital communication network (DCN) for individuals of the population to communicate with each other; receiving, from the member, a report on a level of a psychological or physiological measurement in the DCN; sending, to the member, questions to illicit reflection on factors causing a response to the level of the psychological measurement through communication on the DCN; and receiving a response to the questions on the DCN.

Statement 34. The method of Statement 33, further comprising repeatedly sending additional questions to illicit reflection on factors and receiving additional response to the additional questions on the DCN.

Statement 35. The method of Statement 33, further comprising analyzing communications in the DCN by the member; and generating the questions to illicit the reflection based on the analysis.

Statement 36. The method of Statement 33, wherein the communication related to the reflection is textural.

Statement 37. The method of Statement 33, wherein the communication related to the reflection is a verbal communication.

Statement 38. The method of Statement 37, further comprising translating the verbal communication to a textual communication.

Statement 39. The method of Statement 33, wherein the communication related to the reflection is one of: a visual communication and a graphical communication.

Statement 40. The method of Statement 33, wherein the psychological measurement is stress.

Statement 41. The method of Statement 33, further comprising presenting, to the member, a clickable choice of level of the psychological measurement.

Statement 42. The method of Statement 33, wherein the adverse health condition is one of: chronic systemic inflammation, malaise, low energy, social dysfunction, and a prodromal disease.

Statement 43. The method of Statement 33, wherein the adverse health condition is a disease.

Statement 44. The method of Statement 43, wherein the disease is one of: obesity, diabetes, rheumatoid arthritis, Crohn's disease, psoriasis, eczema, a cardiovascular disease, congestive heart failure, chronic obstructive pulmonary disease, asthma, depression, and anxiety.

Statement 45. The method of Statement 33, wherein the adverse health condition is a health risk.

Statement 46. The method of Statement 45, wherein the health risk is one of: falling, and becoming infected.

Statement 47. The method of Statement 33, wherein communications in the DCN comprise at least one of: one-to-one communications, one-to-many communications, one-to-system communications, system-to-one communications, and system-to-many communications.

Statement 48. The method of Statement 47, wherein the DCN comprises an AI bot configured to derive communications from analysis of communication in the DCN or from biometrics provided to the DCN.

Statement 49. A method to slow progression of adverse health condition of a member of a population, the method comprising: providing a digital communication network (DCN) for individuals of the population to communicate with each other; providing the member with means to show responses to reports of a level of a psychological measurement over time in a graphical format on the DCN; and requesting the member to reflect on causes of the level of the psychological measurement over time on the DCN.

Statement 50. The method of Statement 49, further comprising receiving, from the member, the reports on the level of a psychological measurement over time in the DCN.

Statement 51. The method of Statement 49, wherein the communication related to the reflection is textural.

Statement 52. The method of Statement 49, wherein the communication related to the reflection is a verbal communication.

Statement 53. The method of Statement 52, further comprising translating the verbal communication to a textual communication.

Statement 54. The method of Statement 53, wherein the communication related to the reflection is one of: a visual communication and a graphical communication.

Statement 55. The method of Statement 54, further comprising receiving an annotation on a graphical representation of the level of the psychological measurement over time.

Statement 56. The method of Statement 55, wherein the annotation is a textual annotation.

Statement 57. The method of Statement 55, wherein the annotation is a verbal annotation.

Statement 58. The method of Statement 55, further comprising translating the verbal annotation to a textual annotation.

Statement 59. The method of Statement 55, wherein the annotation is a visual or graphical.

Statement 60. The method of Statement 49, further comprising generating a graphical representation of the level of the psychological measurement over time.

Statement 61. The method of Statement 60, wherein the graphical representation comprises additional psychological measurements.

Statement 62. The method of Statement 49, wherein the psychological measurement is a biomarker derived from one of: saliva, blood, breath, urine, stool and a wearable device.

Statement 63. The method of Statement 49, wherein the psychological measurement is stress.

Statement 64. The method of Statement 49, further comprising presenting, to the member, a clickable choice of level of the psychological measurement.

Statement 65. The method of Statement 49, wherein the adverse health condition is one of: chronic systemic inflammation, malaise, low energy, social dysfunction, and a prodromal disease.

Statement 66. The method of Statement 49, wherein the adverse health condition is a disease.

Statement 67. The method of Statement 66, wherein the disease is one of: obesity, diabetes, rheumatoid arthritis, Crohn's disease, psoriasis, eczema, a cardiovascular disease, congestive heart failure, chronic obstructive pulmonary disease, asthma, depression, and anxiety.

Statement 68. The method of Statement 49, wherein the adverse health condition is a health risk.

Statement 69. The method of Statement 68, wherein the health risk is one of: falling, and becoming infected.

Statement 70. The method of Statement 49, wherein communications in the DCN comprise at least one of: one-to-one communications, one-to-many communications, one-to-system communications, system-to-one communications, and system-to-many communications.

Statement 71. The method of Statement 70, wherein the DCN comprises an AI bot configured to derive communications from analysis of communication in the DCN or from biometrics provided to the DCN.

Statement 72. A method to slow progression of adverse health condition of a member of a population, the method comprising: providing a digital communication network (DCN) for individuals of the population to communicate with each other; observing an occurrence of a psychological or physiological measurement, or one or more levels of a physiological measurement the member communicated to the DCN; sending a communication to the member requesting the member reflect on the psychological or physiological measurement, or a change in levels of the physiological measurement over time; and requesting the member to reflect on causes of the physiological measurement, or the change in levels of the psychological or physiological measurement over time on the DCN.

Statement 73. The method of Statement 72, further comprising observing at least one additional occurrence of the physiological measurement.

Statement 74. The method of Statement 72, wherein observing the physiological measurement comprises observing a level of a physiological measurement.

Statement 75. The method of Statement 72, wherein the communication requesting the member reflect comprises a measurement derived from a wearable device.

Statement 76. The method of Statement 72, wherein the communication requesting the member reflect comprises a measurement derived from lab analysis of a sample provided by the member to the DCN.

Statement 77. The method of Statement 72, wherein observing the occurrence of the physiological measurement comprises observing a combination of different psychological or physiological measurements.

Statement 78. The method of Statement 72, wherein the communication requesting the member reflect comprises an indication of an interrelationship between the combination of the different psychological or physiological measurements.

Statement 79. The method of Statement 72, wherein observing the occurrence of the physiological measurement further comprises observing a combination of the physiological measurement and a psychological measurement.

Statement 80. The method of Statement 79, wherein the communication requesting the member reflect comprises an indication of an interrelationship between the combination of the physiological measurement and the psychological measurement.

Statement 81. The method of Statement 72, wherein the communication related to the reflection is textural.

Statement 82. The method of Statement 72, wherein the communication related to the reflection is a verbal communication.

Statement 83. The method of Statement 82, further comprising translating the verbal communication to a textual communication.

Statement 84. The method of Statement 83, wherein the communication related to the reflection is one of: a visual communication and a graphical communication.

Statement 85. The method of Statement 72, wherein the physiological measurement is a biomarker derived from one of: saliva, blood, breath, urine, stool and a wearable device.

Statement 86. The method of Statement 72, wherein the adverse health condition is one of: chronic systemic inflammation, malaise, low energy, social dysfunction, and a prodromal disease.

Statement 87. The method of Statement 72, wherein the adverse health condition is a disease.

Statement 88. The method of Statement 87, wherein the disease is one of: obesity, diabetes, rheumatoid arthritis, Crohn's disease, psoriasis, eczema, a cardiovascular disease, congestive heart failure, chronic obstructive pulmonary disease, asthma, depression, and anxiety.

Statement 89. The method of Statement 72, wherein the adverse health condition is a health risk.

Statement 90. The method of Statement 89, wherein the health risk is one of: falling, and becoming infected.

Statement 91. The method of Statement 72, wherein communications in the DCN comprise at least one of: one-to-one communications, one-to-many communications, one-to-system communications, system-to-one communications, and system-to-many communications.

Statement 92. The method of Statement 91, wherein the DCN comprises an AI bot configured to derive communications from analysis of communication in the DCN or from biometrics provided to the DCN.

What is claimed is:

1. A method to slow progression of an adverse health condition of an individual member of a population, the method comprising:
providing a digital communication network (DCN) for a plurality of members of a population to communicate with each other;
analyzing communications from an individual member in the DCN;
determining a determined time to transmit an automated communication to the individual member;
transmitting the automated communication to the individual member at the determined time, the automated communication requesting that the individual member respond indicating a current level of at least one of a psychological or physiological measurement; and
receiving a response from the individual member through the DCN.

2. The method of claim 1, wherein the automated communication is transmitted to the individual member two or more times each day and one or more additional responses are received.

3. The method of claim 1, wherein the psychological measurement is stress.

4. The method of claim 1, further comprising presenting, to the member, a clickable choice of level of the psychological or physiological measurement in the automated communication.

5. The method of claim 1, wherein the determined time is based on at least one of when the individual member is active on the DCN, a sleep-wake cycle of the individual member, an eating cycle of the individual member, a work cycle of the individual member, or an activity cycle of the individual member.

6. A method to slow progression of an adverse health condition of an individual member of a population, the method comprising:
providing a digital communication network (DCN) for a plurality of members of a population to communicate with each other;
receiving at least one of a psychological measurement, a physiological measurement, or one or more levels of a physiological measurement of an individual member;
transmitting a communication to the individual member to request the individual member to reflect on a cause of at least one of the one psychological measurement or physiological measurement, or a change in the levels of the physiological measurement; and
receiving the reflection from the individual member.

7. The method of claim 6, wherein the communication requests that the individual member reflects over time on the DCN.

8. The method of claim 6, wherein the communication includes one or more questions to illicit the reflection.

9. The method of claim 8, further comprising:
receiving one or more communications from the individual member in the DCN;
analyzing the one more communications; and
generating the one or more questions based on the analysis of the one or more communications.

10. The method of claim 9, further comprising:
repeatedly sending additional questions to illicit reflection on factors correlating to the at least one psychological measurement, physiological measurement, or the change in the levels of the physiological measurement; and
receiving additional response to the additional questions on the DCN.

11. The method of claim 6, wherein the reflection comprises at least one of a measurement derived from a wearable device, a measurement derived from lab analysis of a sample provided by the individual member.

12. The method of claim 6, wherein the at least one psychological measurement, physiological measurement, or levels of the physiological measurement are received from observing an occurrence of the at least one psychological measurement and/or physiological measurement.

13. The method of claim 12, further comprising observing at least one additional occurrence of the physiological measurement.

14. The method of claim 12, wherein observing the physiological measurement comprises observing a level of a physiological measurement.

15. The method of claim 6, wherein the at least one psychological measurement, physiological measurement, or levels of the physiological measurement are received as a report from the individual member.

16. A method to slow progression of an adverse health condition of an individual member of a population, the method comprising:
providing a digital communication network (DCN) for a plurality of members of a population to communicate with each other;
generating a report of a level of a psychological measurement from an individual member over time in a graphical format;
displaying the report to the individual member;
requesting the individual member to reflect on causes of the level of the psychological measurement over time on the DCN; and
receiving a reflection from the individual member on the DCN.

17. The method of claim 16, further comprising:
receiving an updated level of the psychological measurement from the individual member over an updated time;
updating the report of the level of the psychological measurement in the graphical formal based on the updated level of the psychological measurement and the reflection; and
displaying the updated report to the individual member.

18. The method of claim 16, wherein the reflection is at least one of textual, verbal, a verbal communication converted to a textual communication, a visual communication, or a graphical communication.

19. The method of claim 16, wherein the report is generated by the individual member.

20. The method of claim 16, further comprising receiving an annotation on the graphical format of the level of the psychological measurement over time.
